(19) United States
(12) Patent Application Publication
(10) Pub. No.: US 2012/0035177 A1
(43) Pub. Date: Feb. 9, 2012

(54) TABLET FORMULATION FOR P38 INHIBITOR AND METHOD

(75) Inventors: Manisha M. Dali, Bridgewater, NJ (US); Charles E. Dahlheim, Lawrenceville, NJ (US); Vijay H. Naringrekar, Princeton, NJ (US); Gary McGeorge, East Windsor, NJ (US)

(73) Assignee: Bristol-Myers Squibb Company, Princeton, NJ (US)

(21) Appl. No.: 13/263,877

(22) PCT Filed: Apr. 15, 2010
(86) PCT No.: PCT/US10/31138

§ 371 Date: Oct. 11, 2011

Related U.S. Application Data
(60) Provisional application No. 61/169,873, filed on Apr. 16, 2009.

(51) Int. Cl. A61K 31/53; A61P 11/00; A61P 29/00

(52) U.S. Cl. 514/243

(57) ABSTRACT

A tablet formulation is provided which includes a medicament which is a pharmaceutically acceptable salt of a p38 inhibitor, such as the p38 HCl salt of the structure Formula (I) and which has good physical stability when stored at up to 25 C./60% RH in closed containers with desiccant. The tablet formulation will contain crospovidone as a tablet disintegrant, which, unlike croscarmellose sodium, will not cause disproportionation of the HCl salt to the free base of P38 inhibitor and thus will have acceptable dissolution properties even after storage at room temperature.

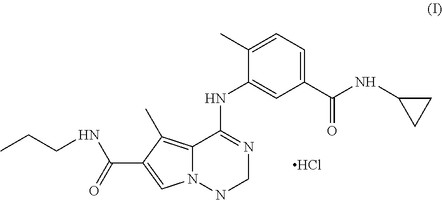

### FIELD OF THE INVENTION

The present invention relates to a tablet formulation which includes a medicament which is a p38 inhibitor, and to a method for preparing such tablet formulation.

### BACKGROUND OF THE INVENTION

The compound of the structure

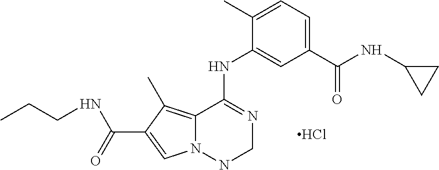

(hereinafter the p38 inhibitor HCl salt) is an orally active nonhygroscopic crystalline p38 α-map kinase inhibitor, which is a therapeutic agent for treatment of rheumatoid arthritis and chronic obstructive pulmonary disease (COPD), is disclosed in U.S. application Ser. No. 11/398,102 filed Apr. 4, 2006, the disclosure of which is incorporated herein by reference.

The above p38 inhibitor has been found to have good physical stability upon storage at room temperature without need for refrigeration. However, when formulated into various tablet dosage forms, which include the disintegrant croscarmellose sodium, it has been found that the tablets, when stored above 2 to 8° C., harden and exhibit a dissolution slow-down that progresses to the point where incomplete dissolution occurs. The slow-down in dissolution of the tablet formulation containing the p38 inhibitor HCl salt has been found to be proportional to exposure to high temperature and humidity thereby necessitating storage under refrigeration.

Findings of studies conducted to determine the cause of the dissolution slow-down of tablets containing the p38 inhibitor HCl salt at elevated temperatures indicate that it is caused by disproportionation of the p38 inhibitor HCl salt to the free base. This is attributed to a moisture-mediated excipient incompatibility of the p38 inhibitor HCl salt with the croscarmellose sodium disintegrant present in the tablets.

Accordingly, it is seen that there clearly is a need for a stable tablet formulation containing the p38 inhibitor HCl salt which has improved physical stability over previous tablet formulations containing croscarmellose sodium and which can be stored at room temperature and 60% relative humidity in closed containers without disproportionation of the p38 inhibitor HCl salt to the free base.

It has been found that the tablet formulation of the invention which includes the p38 inhibitor HCl salt, where crospovidone is used as the disintegrant, has good physical stability when stored at up to 25° C./60% relative humidity in closed containers with desiccant.

Crospovidone used as the tablet disintegrant, unlike croscarmellose sodium, will not cause disproportionation of the p38 inhibitor HCl salt to the free base and thus the tablet formulation of the invention will have acceptable dissolution properties without need for refrigeration.

### BRIEF STATEMENT OF THE INVENTION

In accordance with the present invention a pharmaceutical formulation in the form of a tablet is provided which includes:
a) a p38 inhibitor in the form of a pharmaceutically acceptable salt;
b) one or more tablet excipients; and
c) as a tablet disintegrant crospovidone.

In a preferred embodiment, the pharmaceutical formulation of the invention is in the form of a tablet which includes:
a) a p38 inhibitor in the form of a pharmaceutically acceptable salt;
b) one or more fillers or more bulking agents;
c) one ore more buffering agents;
d) one or more wetting agents or surfactants;
e) one or more glidants;
f) one or more lubricants; and
g) as a tablet disintegrant crospovidone.

The p38 inhibitor which will preferably be employed as the medicament in the tablet formulation of the invention is the compound of the structure in the form of its HCl salt:

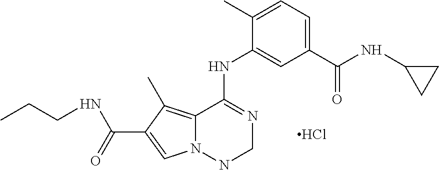

hereinafter referred to as “the p38 inhibitor HCl salt” or “p38 inhibitor”.

The tablet formulation of the invention is useful in the treatment of mammals such as humans and cats for rheumatoid arthritis and chronic obstructive pulmonary disease (COPD).

### DETAILED DESCRIPTION OF THE INVENTION

The tablet formulation of the invention will include conventional pharmaceutical excipients to enable formation of a pharmaceutically acceptable solid tablet. The tablet formulation may be in the form of a tablet, bead, beadlet, or pill, all of the above being collectively referred to as a tablet formulation.

The tablet of the invention will contain medicament, preferably the p38 inhibitor HCl salt, in an amount within the range from 0.1 to 50% by weight and preferably from 5 to 45% by weight and more preferably from 10 to 25% by weight, all based on the weight of the finished tablet.

In addition to the p38 inhibitor HCl salt, the tablet formulation of the invention will preferably contain
a) at least one bulking agent or filler which may serve as a compressibility aid;
b) at least one buffering agent which serves to maintain microenvironmental pH during dissolution in gastric juices below 3;
c) at least one wetting agent or surfactant;
d) at least one flow aid arid/or glidant;
e) at least one lubricant; and
f) a tablet disintegrant which is crospovidone and not croscarmellose sodium.

The tablet formulation of the invention will preferably include
a) the bulking agent or filler in an amount within the range from 25 to 90% by weight, preferably from 45 to 65% by weight;
b) the buffering agent in an amount within the range from 1 to 20% by weight, preferably from 10 to 15% by weight;
c) the wetting agent or surfactant in an amount within the range from 1 to 5% by weight, preferably from 2 to 5% by weight;
d) the flow aid or glidant in an amount within the range from 0.5 to 5% by weight, preferably from 0.5 to 2% by weight;
e) the lubricant in an amount within the range from 0.5 to 1.5% by weight, preferably from 0.6 to 1% by weight; and
f) the crospovidone disintegrant in an amount within the range from 4 to 10% by weight, and preferably from 6 to 8% by weight;
   all of the above % by weight being based on the total weight of the tablet formulation.

It is preferred that
a) the bulking agent is selected from microcrystalline cellulose and lactose, and more preferably a combination of microcrystalline cellulose and lactose;
b) the buffering agent is selected from succinic acid and tartaric acid, more preferably succinic acid;
c) the wetting agent or surfactant is sodium lauryl sulfate;
d) the flow aid or glidant is silicon dioxide or Cab-o-Sil (fumed silica);
e) the lubricant is magnesium stearate; and
f) the disintegrant is crospovidone and not croscarmellose sodium.

The tablet formulation of the invention can be prepared by a variety of processes and order of addition of excipients. The utility of these formulations is not limited to a specific dosage form or manufacturing process. Tablet may be manufactured by dry granulation, direct blending or any other pharmaceutically acceptable process.

In accordance with the present invention, a preferred method is provided for preparing the tablet of the invention which includes the steps of blending the one or more excipients such as bulking agents, glidant, buffering and wetting agent. Buffering agent and p38 inhibitor are added to the blend. Additional bulking agent, and disintegrant are then mixed with the blend. A lubricant will be preferably added to the blend to facilitate tablet formulation. The resulting blend is then compacted and sized to form an intragranular portion which is mixed with an extragranular portion of crospovidone, lubricant, and flow-aid. The resulting granulation is compressed into tablets of the invention.

The disintegrant crospovidone (homopolymer of cross-linked N-vinyl-2-pyrrolidone) is marketed under the tradename POLYPLASDONE® XL (average particle size 100 microns) and POLYPLASDONE® XL-10 (average particle size 30 microns), both available from ISP Technologies, Inc.

The crospovidone employed in preparing the tablets of the invention in each of the intragranular portion and the extragranular portion may be the same, namely the POLYPLASDONE® XL crospovidone. However, if desired, the crospovidone employed in the intragranular portion may be the POLYPLASDONE® XL-10 and the crospovidone employed in the extragranular portion may be the POLYPLASDONE® XL.

The bulking agents or fillers will be present in the tablet formulations of the invention in an amount within the range from 1 to 95% by weight, preferably from 25 to 90% by weight of the tablet formulation, and more preferably from 45 to 65% by weight of the tablet formulation. Examples of bulking agents or fillers suitable for use herein include, but are not limited to, cellulose derivatives such as microcrystalline cellulose or wood cellulose, lactose, sucrose, starch, pregelatinized starch, dextrose, mannitol, fructose, xylitol, sorbitol, corn starch, modified corn starch, dextrin/dextrates, maltodextrin, compressible sugars, and other known bulking agents or fillers, and/or mixtures of two or more thereof, preferably a combination of microcrystalline cellulose and lactose, preferably from 20 to 60%, more preferably 40 to 50% by weight microcrystalline cellulose and from 5 to 30%, more preferably 5 to 15% by weight lactose, based on the total weight of the tablet formulation.

The buffering agent will provide an acidic medium during dissolution and will be present in an amount within the range from 1 to 20%, preferably from 10 to 15% by weight of the tablet formulation so that the tablet formulation of the invention during dissolution in the body will have a pH of less than 3, preferably less than 2.5 to ensure acceptable dissolution of the p38 inhibitor. Examples of buffering agents suitable for use herein include, but are not limited to, succinic acid, tartaric acid, acetic acid, citric acid, fumaric acid, hydrochloric acid, ascorbic acid, malic acid, maleic acid, and, preferably succinic acid.

The wetting agent or surfactant will be used in the tablet formulation of the invention to aid in solubilizing the p38 inhibitor HCl salt and will be present in an amount within the range from 1 to 5% by weight, preferably from 2 to 5% by weight of the tablet formulation of the invention. Examples of wetting agents or surfactants suitable for use herein include, but are not limited to, sodium lauryl sulfate or Poloxamer 188 (PLURONIC® F68; polyethylene-polypropylene glycol HO(C₂H₄O)a(C₃H₆O)b(C₂H₄O)aH Av. M.W. 8400) with sodium lauryl sulfate being preferred.

The glidant or flow aid will be used in the tablet formulation of the invention to aid powder blend flow from manufacturing equipment into the tablet press and dies and also to reduce friction during compression and will be present in an amount within the range from 0.5 to 5% by weight, preferably from 0.5 to 2% by weight based on the tablet formulation of the invention. Examples of glidants or flow aids suitable for use in the tablet formulation of the invention include, but are not limited to, silicon dioxide, colloidal silica, fumed silica, cornstarch, talc, calcium silicate, magnesium silicate, and silicon hydrogel, with silicon dioxide being preferred.

The lubricant will be used in the tablet formulation of the invention to reduce sticking to punches and dies, and reduce friction during tablet compression and will be present in an amount within the range from 0.5 to 1.5% by weight, preferably from 0.6 to 1% by weight of the tablet formulation of the invention. Examples of lubricants suitable for use herein include, but are not limited to, magnesium stearate, sodium stearyl fumarate, carnauba wax, palmitic acid, calcium stearate, mineral oil, stearic acid and zinc stearate with magnesium stearate being preferred.

As indicated, the tablet formulation of the invention will include as a tablet disintegrant crospovidone (and not croscarmellose sodium) in an amount within the range from 2 to 10% by weight, preferably from 6 to 8% by weight of the tablet formulation of the invention.

In addition, the tablet formulation of the invention may include other conventional excipients and ingredients such as antioxidants, colorants, flavorants, sweetening agents, antiadherents, binders, and diluents.

The formulation of the invention may optionally include an outer protective layer which will include up to 95% of coating layer polymer based on the weight of the protective coating layer, and a coating solvent such as ethanol or isopropyl alcohol which is used for processing, and is removed by drying. The coating layer polymer may be hydroxypropyl methylcellulose, polyvinyl alcohol (PVA), ethyl cellulose, methacrylic polymers or hydroxypropyl cellulose, preferably PVA. The coating layer may also optionally include a plasticizer such as triacetin, diethyl phthalate, tributyl sebacate or polyethylene glycol (PEG), preferably PEG; and an anti-adherent or glidant such as talc, fumed silica or magnesium stearate, and opacifying agent such as titanium dioxide. The coating layer may also include iron oxide based colorants. The coating material is commercially available under the trade name OPADRY® HP or OPADRY® II white.

In a preferred embodiment of the invention, an outer protective coating layer will be coated over the tablet of the invention and will function as a protective layer. The protective coating layer may optionally include a coating polymer and colorants to differentiate tablets of various strengths.

Preferred are tablet formulations as set out below

Optimized Tablet Formulations

| | 10 mg Tablet | | 100 mg Tablet | |
|---|---|---|---|---|
| Ingredient | mg/tablet | Wt % | mg/tablet | Wt % |
| p38 Inhibitor | 10.89^{(a)} | 10.89 | 108.92^{(a)} | 27.23 |
| Microcrystalline Cellulose, NF | 48.5 | 48.5 | 169.08 | 42.27 |
| Lactose Anhydrous, NF | 10.0 | 10.0 | 40.00 | 10.00 |
| Succinic Acid | 15.86 | 15.86 | 40.00 | 10.0 |
| Crospovidone, NF | 7.0 | 7.0 | 28.00 | 7.00 |
| Sodium Lauryl Sulfate | 5.0 | 5.0 | 8.00 | 2.0 |
| Silicon Dioxide | 2.0 | 2.0 | 2.00 | 0.50 |
| Magnesium Stearate | 0.75 | 0.75 | 4.00 | 1.0 |

^{(a)}based on 100% purity.

Other 100 mg Potency Tablet Formulations of p38 Inhibitor

100 mg Tablet

| Ingredient | Wt % | Wt % |
|---|---|---|
| p38 Inhibitor | 27.23^{(a)} | 27.23^{(a)} |
| Microcrystalline Cellulose | 41.77 | 42.27 |
| Lactose Anhydrous | 10.0 | 10.0 |
| Succinic Acid | — | 10.0 |
| Tartaric Acid | 10.0 | — |
| Crospovidone | 7.00 | 7.00 |
| Sodium Lauryl Sulfate | 2.0 | 2.0 |
| Silicon Dioxide | 1.0 | — |
| Cab-o-Sil | — | 0.5 |
| Magnesium Stearate | 1.0 | 1.0 |

^{(a)}based on 100% purity

The following Examples are illustrative of preferred embodiments of the present invention.

### EXAMPLE 1

10 mg Potency Tablets

| | | 10 mg potency | Per 350 g |
|---|---|---|---|
| Ingredients: 10 mg potency tablets | | (wt %) | batch (g) |
| Intra- | p38 Inhibitor | 10.89 | 38.12 |
| granular | Lactose Anhydrous | 10.00 | 35.0 |
| | (bulking agent) | | |
| | Microcrystalline Cellulose | 48.50 | 169.75 |
| | (bulking agent) | | |
| | Sodium Lauryl Sulfate | 5.00 | 17.5 |
| | (wetting agent) | | |
| | Succinic Acid (buffer) | 15.86 | 55.51 |
| | Silicon Dioxide (flow aid) | 2.00 | 7.00 |
| | Crospovidone (disintegrant) | 2.00 | 7.00 |
| | Magnesium Stearate (lubricant) | 0.375 | 1.31 |
| Extra- | Crospovidone | 5.00 | 17.50 |
| granular | Magnesium Stearate | 0.375 | 1.31 |

| | | |
|---|---|---|
| Total | 100.00 | 350.00 |

The above tablet formulation of the invention was prepared employing the following procedure,

Lactose anhydrous, sodium lauryl sulfate, and silicon dioxide (all 20 mesh or less particle size) were combined in a bin blender and mixed for 10 minutes at approximately 25 rpm. The p38 inhibitor (20 mesh or less) and succinic acid (100 mesh or less) were added to the blender and mixed for 10 minutes at approximately 25 rpm. The microcrystalline cellulose was added to the blender and the contents mixed for 10 minutes at approximately 25 rpm. The intragranular portion of the crospovidone was added to the blender and the contents mixed for 10 minutes at approximately 25 rpm. The intragranular portion of the magnesium stearate was added to the blender and the contents mixed for 3 minutes at approximately 25 rpm. The resulting powder blend was roller compacted in a Vector Mini-Freund roller compactor using a 12 rpm screw speed, a 4 rpm roller speed, and a 10 to 15 kg force per square centimeter roller pressure to produce compacted ribbons which were sized to pass through an 18 mesh screen using an oscillator to produce a granulation. The granulation was added to the bin blender and the extragranular portion of the crospovidone was added. The contents of the blender were mixed for 10 minutes at approximately 25 rpm. The extragranular portion of the magnesium stearate was added to the blender and the contents mixed for 3 minutes at approximately 25 rpm. The resulting granulation was compressed into tablets of approximately 100 mg gross weight using an appropriate tablet press and ¼ inch round standard-concave tooling to achieve a target tablet hardness of approximately 7 Strong-Cobb Units (SCU), with an acceptable range of 5 to 10 SCU.

### EXAMPLE 2

100 mg Potency Tablets

| | | 100 mg potency | Per 5000 g |
|---|---|---|---|
| Ingredients: 100 mg potency tablets | | (wt %) | batch (g) |
| Intra- | p38 Inhibitor | 27.23 | 1361.5 |
| granular | Lactose Anhydrous | 10 | 500 |
| | (bulking agent) | | |
| | Microcrystalline Cellulose | 42.27 | 2113.5 |
| | (bulking agent) | | |
| | Sodium Lauryl Sulfate | 2 | 100 |
| | (wetting agent) | | |
| | Succinic Acid (buffer) | 10.0 | 500 |
| | Crospovidone (disintegrant) | 2 | 100 |
| | Magnesium Stearate (lubricant) | 0.50 | 25 |
| Extra- | Silicon Dioxide (flow aid) | 0.50 | 25 |
| granular | Crospovidone | 5 | 250 |
| | Magnesium Stearate | 0.50 | 25 |

| | | |
|---|---|---|
| Total | 100.00 | 5000 |

The above tablet formulation of the invention was prepared employing the following procedure.

Lactose anhydrous and sodium lauryl sulfate (all 20 mesh or less) in a bin blender of appropriate size were mixed for 10 minutes at approximately 25 rpm. The p38 inhibitor and succinic acid were added to the blender and the contents mixed for 10 minutes at approximately 25 rpm. Microcrystalline cellulose was added to the blender and the contents mixed for 10 minutes at approximately 25 rpm. The intragranular portion of the crospovidone was added to the blender and the contents mixed for 10 minutes at approximately 25 rpm. The intragranular portion of the magnesium stearate was added to the blender and the contents mixed for 3 minutes at approximately 25 rpm. The resulting powder blend was roller compacted in a Vector Mini-Freund roller using a 12 rpm screw speed, a 4 rpm roller speed, and a 13 to 15 kg force per square centimeter roller pressure to form compacted ribbons. The compacted ribbons were sized through an 18 mesh screen using an oscillator and the resulting granulation returned to the blender. The extragranular portion of the crospovidone and silicone dioxide were added to the blender and the contents mixed for 10 minutes at approximately 25 rpm. The extragranular portion of the magnesium stearate was added to the blender and the contents mixed for 3 minutes at approximately 25 rpm. The resulting granulation was compressed into tablets of approximately 400 mg gross weight using an appropriate tablet press and 13/32 inch round standard-concave tooling. The compression force was adjusted to achieve a target tablet hardness of approximately 10-12 Strong-Cobb Units (SCU), with an acceptable range of 9 to 13 SCU.

### EXAMPLE 3

The Example 3 100 mg tablet formulation containing crospovidone as a disintegrant and having a target tablet hardness of approximately 10-12 Strong-Cobb Units (SCU), with an acceptable range of 9 to 13 SCU, was prepared as described in Example 2.

The Example 3 tablet (100 mg) was tested against a similar 100 mg tablet formulation having a target tablet hardness of approximately 12 Strong-Cobb Units (referred to as the Comparator) containing croscarmellose sodium as the disintegrant (in place of crospovidone) (as set out in Table A below) to determine dissolution properties of each tablet formulation subjected to similar temperature and relative humidity. The results of such dissolution tests on the Example 3 tablet and the comparator tablet are set out below in Table B.

TABLE A
100 mg Tablet

| | | Comparator | Example 3 |
|---|---|---|---|
| Ingredients | | (wt %) | (wt %) |
| Intra- | p38 Inhibitor | 27.23 | 27.23 |
| granular | Anhydrous Lactose, NF | 10.50 | 10.00 |
| | Microcrystalline Cellulose, NF | 35.77 | 38.00 |
| | Sodium Lauryl Sulfate, NF | 5.00 | 2.00 |
| | Silicon Dioxide, NF | 0.50 | 0 |
| | Succinic Acid, FCC | 10.00 | 14.27 |
| | Crospovidone | 0 | 2.0 |
| | (POLYPLASDONE ® XL-10) | | |
| | Croscarmellose Sodium, NF | 5.00 | 0 |
| | Magnesium Stearate, NF | 0.50 | 0.50 |
| Extra- | Silicon Dioxide | 0 | 0.50 |
| granular | Croscarmellose Sodium, NF | 5.00 | 0 |
| | Crospovidone | 0 | 5.00 |
| | (POLYPLASDONE ® XL) | | |
| | Magnesium Stearate | 0.50 | 0.50 |

| | | |
|---|---|---|
| Total (%) | 100 | 100 |

The Comparator tablets were prepared in a manner similar to that described in Example 3 except that croscarmellose sodium was used in place of crospovidone.

TABLE B
Tablets Containing p38 Inhibitor HCl Salt Dissolution
Medium - H₂O, 0.05N HCl, 1% Sodium Lauryl Sulfate

| | Storage Condition | | Storage Condition | |
|---|---|---|---|---|
| | 25° C./60% RH, | | 40° C./75% RH, | |
| | Closed Container | | Closed Container | |
| | % p38 Inhibitor Dissolved | | % p38 Inhibitor Dissolved | |
| Storage Time | Example 3 | Comparator | Example 3 | Comparator |
| (weeks) | Tablet | Tablet | Tablet | Tablet |

a) 10 Minutes Dissolution Time (min.)

| | | | | | |
|---|---|---|---|---|---|
| 2 | weeks | — | 60% | 72% | 30% |
| 4 | weeks | 68% | 46% | 67% | 18% |
| 8 | weeks | 71% | — | 63% | — |
| 12-13 | weeks | 69% | 26% | — | 5% |
| 26 | weeks | 69% | 21% | — | 4% |

b) 20 Minutes Dissolution Time (min.)

| | | | | | |
|---|---|---|---|---|---|
| 2 | weeks | — | 88% | 92% | 69% |
| 4 | weeks | 91% | 84% | 89% | 52% |
| 8 | weeks | 92% | — | 86% | — |
| 12-13 | weeks | 92% | 70% | — | 12% |
| 26 | weeks | 90% | 55% | — | 8% |

c) 30 Minutes Dissolution Time (min.)

| | | | | | |
|---|---|---|---|---|---|
| 2 | weeks | — | 93% | 98% | 87% |
| 4 | weeks | 98% | 92% | 97% | 78% |
| 8 | weeks | 99% | — | 95% | — |
| 12-13 | weeks | 100% | 88% | — | 23% |
| 26 | weeks | 98% | 81% | — | 13% |

d) 45 Minutes Dissolution Time (min.)

| | | | | | |
|---|---|---|---|---|---|
| 2 | weeks | — | 96% | 101% | 95% |
| 4 | weeks | 101% | 97% | 101% | 88% |
| 8 | weeks | 102% | — | 95% | — |
| 12-13 | weeks | 104% | 96% | — | 38% |
| 26 | weeks | 101% | 90% | — | 20% |

e) 60 Minutes Dissolution Time (min.)

| | | | | | |
|---|---|---|---|---|---|
| 2 | weeks | — | 97% | 101% | 97% |
| 4 | weeks | 102% | 98% | 101% | 91% |
| 8 | weeks | 102% | — | 102% | — |
| 12-13 | weeks | 104% | 97% | — | 46% |
| 26 | weeks | 102% | 93% | — | 24% |

The results in the above table show that crospovidone containing tablets (Example 3) stored at 25° C./60% RH for up to 26 weeks in closed containers did not demonstrate the dissolution slow-down seen in the croscarmellose sodium containing Comparator tablets.

The dissolution slow-down in the Comparator tablets is attributed to conversion of the p38 inhibitor HCl salt to the free base.

What is claimed is:

1. A pharmaceutical formulation in the form of a tablet comprising:
a) a p38 kinase inhibitor in the form of a pharmaceutically acceptable salt;
b) one or more bulking agents;
c) one or more buffering agents; and
d) crospovidone as a tablet disintegrant;
said tablet formulation having acceptable physical stability and chemical stability when stored at room temperature and 60% relative humidity.

2. The pharmaceutical formulation as defined in claim 1 which is substantially free of croscarmellose sodium.

3. The pharmaceutical formulation as defined in claim 1 wherein the p38 inhibitor has the structure

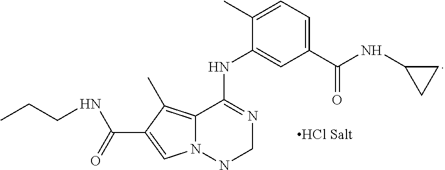

4. The pharmaceutical formulation as defined in claim 1 comprising, in addition:
a) one or more surfactants;
b) one or more glidants; and
c) one or more lubricants.

5. The pharmaceutical formulation as defined in claim 1 comprising:
a) a p38 kinase inhibitor HCl salt having the structure 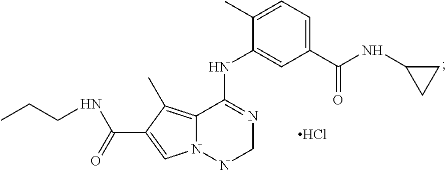
b) one or more bulking agents selected from the group consisting of microcrystalline cellulose or wood cellulose, lactose, sucrose, starch, pregelatinized starch, dextrose, mannitol, fructose, xylitol, sorbitol, corn starch, modified corn starch, calcium carbonate, calcium phosphate, dicalcium phosphate, calcium sulfate, dextrin/dextrates, maltodextrin, compressible sugars, and mixtures thereof;
c) one or more surfactants selected from the group consisting of sodium lauryl sulfate or polyethylene-polypropylene glycol (Poloxamer 188);
d) one or more buffering agents selected from the group consisting of succinic acid, tartaric acid, citric acid, acetic acid, fumaric acid, hydrochloric acid, ascorbic acid, malic acid and maleic acid;
e) one or more glidants selected from the group consisting of silicon dioxide, colloidal silica, fumed silica, cornstarch, talc, calcium silicate, magnesium silicate, and silicon hydrogel; and
f) one or more tablet lubricants selected from the group consisting of magnesium stearate, calcium stearate, mineral oil, stearic acid, and zinc stearate.

6. The pharmaceutical formulation as defined in claim 1 wherein:
a) the p38 inhibitor is present in an amount within the range from 0.1 to 50% by weight;
b) the bulking agent is present in an amount within the range from 25 to 95% by Weight;
c) the crospovidone disintegrant is present in an amount within the range from 2 to 10% by weight;
d) the lubricant is present in an amount within the range from 0.5 to 1.5% by weight; and
e) the glidant is present in an amount within the range from 0.5 to 5% by weight;
all of the above % are based on the total weight of the finished tablet.

7. The pharmaceutical formulation as defined in claim 1 wherein:
a) the bulking agent is selected from the group consisting of microcrystalline cellulose, lactose or a combination thereof; or
b) the buffering agent is selected from the group consisting of succinic acid and tartaric acid; or
c) the wetting agent is sodium lauryl sulfate; or
d) the flow aid is silicon dioxide or fumed silica; or
e) the lubricant is magnesium stearate; and
f) the disintegrant is crospovidone.

8. The pharmaceutical formulation as defined in claim 1 which comprises:
a) the p38 inhibitor which is 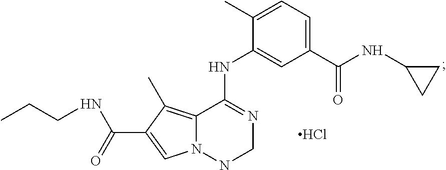
b) lactose anhydrous;
c) microcrystalline cellulose;
d) sodium lauryl sulfate;
e) succinic acid;
f) silicon dioxide;
g) crospovidone; and
h) magnesium stearate.

9. The pharmaceutical formulation as defined in claim 1 having the following composition:

| | 10 mg Tablet | | 100 mg Tablet | |
|---|---|---|---|---|
| Ingredient | mg/tablet | Wt % | mg/tablet | Wt % |
| p38 Inhibitor | 10.89^{(a)} | 10.89 | 108.92 | 27.23^{(a)} |
| Microcrystalline Cellulose, NF | 48.5 | 48.5 | 169.08 | 42.27 |
| Lactose Anhydrous, NF | 10.0 | 10.0 | 40.00 | 10.00 |
| Succinic Acid | 15.86 | 15.86 | 40.00 | 10.0 |
| Crospovidone, NF | 7.0 | 7.0 | 28.00 | 7.00 |
| Sodium Lauryl Sulfate | 5.0 | 5.0 | 8.00 | 2.0 |
| Silicon Dioxide | 2.0 | 2.0 | 2.00 | 0.50 |
| Magnesium Stearate | 0.75 | 0.75 | 4.00 | 1.0 |

^{(a)}based on 100% purity

10. The pharmaceutical formulation as defined in claim 1 having the following composition:
100 mg Tablet

| Ingredient | Wt % | Wt % |
|---|---|---|
| p38 Inhibitor | 27,23 | 27.23 |
| Microcrystalline Cellulose | 41.77 | 42.27 |
| Lactose Anhydrous | 10.0 | 10.0 |
| Succinic Acid | — | 10.0 |
| Tartaric Acid | 10.0 | — |
| Crospovidone | 7.00 | 7.00 |
| Sodium Lauryl Sulfate | 2.0 | 2.0 |
| Silicon Dioxide | 1.0 | — |
| Cab-o-Sil | — | 0.5 |
| Magnesium Stearate | 1.0 | 1.0 |

11. The pharmaceutical formulation as defined in claim 1 having the following composition:

| | | 10 mg potency | Per 350 g |
|---|---|---|---|
| Ingredients: 10 mg potency tablets | | (wt %) | batch (g) |
| Intra- | p38 Inhibitor | 10.89 | 38.12 |
| granular | Lactose Anhydrous | 10.00 | 35.0 |
| | (bulking agent) | | |
| | Microcrystalline Cellulose | 48.50 | 169.75 |
| | (bulking agent) | | |
| | Sodium Lauryl Sulfate | 5.00 | 17.5 |
| | (wetting agent) | | |
| | Succinic Acid (buffer) | 15.86 | 55.51 |
| | Silicon Dioxide (flow aid) | 2.00 | 7.00 |
| | Crospovidone (disintegrant) | 2.00 | 7.00 |
| | Magnesium Stearate (lubricant) | 0.375 | 1.31 |
| Extra- | Crospovidone | 5.00 | 17.50 |
| granular | Magnesium Stearate | 0.375 | 1.31 |

12. The pharmaceutical formulation as defined in claim 1 having the following composition:

| | | 100 mg potency | Per 5000 g |
|---|---|---|---|
| Ingredients: 100 mg potency tablets | | (wt %) | batch (g) |
| Intra- | p38 Inhibitor | 27.23 | 1361.5 |
| granular | Lactose Anhydrous | 10 | 500 |
| | (bulking agent) | | |
| | Microcrystalline Cellulose | 42.27 | 2113.5 |
| | (bulking agent) | | |
| | Sodium Lauryl Sulfate | 2 | 100 |
| | (wetting agent) | | |
| | Succinic Acid (buffer) | 10.0 | 500 |
| | Crospovidone (disintegrant) | 2 | 100 |
| | Magnesium Stearate (lubricant) | 0.50 | 25 |
| Extra- | Silicon Dioxide (flow aid) | 0.50 | 25 |
| granular | Crospovidone | 5 | 250 |
| | Magnesium Stearate | 0.50 | 25 |

13. The pharmaceutical formulation as defined in claim 1 having the following composition:
100 mg Tablet

| Ingredients 100 mg Potency Tablets | | Wt % |
|---|---|---|
| Intragranular | p38 Inhibitor | 27.23 |
| | Anhydrous Lactose, NF | 10.00 |
| | Microcrystalline Cellulose, NF | 38.00 |
| | Sodium Lauryl Sulfate, NF | 2.00 |
| | Succinic Acid, FCC | 14.27 |
| | Crospovidone | 2.0 |
| | (POLYPLASDONE ® XL-10) | |
| | Magnesium Stearate, NF | 0.50 |
| Extragranular | Silicon Dioxide | 0.50 |
| | Crospovidone | 5.00 |
| | (POLYPLASDONE ® XL) | |
| | Magnesium Stearate | 0.50 |
| | |
|---|---|
| Total (%) | 100 |
